(19) United States
(12) Patent Application Publication
(10) Pub. No.: US 2023/0015065 A1
(43) Pub. Date: Jan. 19, 2023

(54) MEDICAL PUNCTURE NEEDLE

(71) Applicant: TERUMO KABUSHIKI KAISHA, Tokyo (JP)

(72) Inventors: Yoichiro IWASE, Hadano-shi (JP); Minami MAEKAWA, Kawasaki-shi (JP); Yasuyuki YOSHIDA, Tokyo (JP)

(73) Assignee: TERUMO KABUSHIKI KAISHA, Tokyo (JP)

(21) Appl. No.: 17/946,090

(22) Filed: Sep. 16, 2022

Related U.S. Application Data
(63) Continuation of application No. PCT/JP2021/009411, filed on Mar. 10, 2021.

(30) Foreign Application Priority Data
Mar. 24, 2020 (JP) 2020-052001

(51) Int. Cl. A61M 5/32

(52) U.S. Cl. CPC A61M 5/3286; A61M 5/329

(57) ABSTRACT

A medical puncture needle is configured to reliably administer a sufficient amount of a liquid medicine to thin tissue. The medical puncture needle includes: a main body having a cylindrical shape and having inside a through hole; a blade surface that has an annular shape and that is cut into a tapered shape so that a thickness of the main body reduces toward a tip part of the main body; and a blade edge that has an annular shape, the blade edge being formed at a tip of the blade surface so as to extend along a plane orthogonal to a central axis of the main body.

### CROSS-REFERENCES TO RELATED APPLICATIONS

This application is a continuation of International Application No. PCT/JP2021/009411 filed on Mar. 10, 2021, which claims priority to Japanese Patent Application No. 2020-052001 filed on Mar. 24, 2020, the entire content of both of which is incorporated herein by reference.

### TECHNOLOGICAL FIELD

The present invention generally relates to a medical puncture needle for administering medicine into a living body.

### BACKGROUND DISCUSSION

When medicine is administered to thin tissue in a living body such as a gastrointestinal mucous membrane, a posterior eye segment, or a mucous membrane of a nose using a common injection needle, an inclined blade surface may penetrate the tissue to cause a leakage of the liquid medicine to the outside of the tissue, or the injection needle may penetrate through the thin tissue, because the tissue is as thin as 1 mm or less. Therefore, there is a problem that a sufficient amount of liquid medicine cannot be reliably administered to the target tissue.

In order to address such a problem, U.S. Pat. No. 8,562,589 discloses a method of administering medicine into a gastrointestinal mucous membrane using a puncture needle in which the medicine is applied to the tip of a microneedle.

### SUMMARY

However, the technology disclosed in U.S. Pat. No. 8,562,589 has a problem in that the amount of the medicine applied to the puncture needle is small, and so a sufficient amount of the medicine cannot be efficiently administered.

The medical puncture needle disclosed here is configured to reliably administer a sufficient amount of a liquid medicine to a thin tissue.

One aspect of the disclosure here provides a medical puncture needle including: medical puncture needle comprising: a main body possessing a central axis and a cylindrical shape, the main body including a through hole extending throughout the main body from a proximal end of the main body to a distal end of the main body; a blade surface that has an annular shape and that is tapered so that a thickness of the main body decreases toward the distal end of the main body; and a blade edge that has an annular shape, the blade edge being at a tip of the blade surface and being configured so that the blade edge extends along a plane orthogonal to the central axis of the main body.

According to the medical puncture needle of the above aspect, it is possible to efficiently administer a large amount of liquid medicine to a thin tissue.

According to another aspect, a medical puncture needle for puncturing tissue of a living body to administer liquid medicine comprises: a cylindrically-shaped main body possessing a central axis, the main body including a through hole that extends axially throughout the main body from a proximal end of the cylindrically-shaped main body to an opposite axial end of the cylindrically-shaped main body, the main body including an inner surface that surrounds the through hole; the main body including a distal end portion that terminates at the distal end of the main body, an annular-shaped inclined surface at the distal end portion of the main body, the inclined surface being inclined relative to the central axis so that the distal end portion of the main body is tapered and a thickness of the main body decreases toward the distal end of the main body; the inclined surface terminating at a blade edge that is first brought into contact with the tissue of the living body during the puncture of the tissue of the living body to administer the liquid medicine; and a liquid medicine groove in the inner surface of the main body defining a flow path for guiding the liquid medicine to the distal end portion of the main body when the through hole is closed as a result of coring during the puncture of the tissue.

Another aspect involves a method of administering liquid medicine to tissue of a living body. The method comprises moving a medical puncture needle toward the tissue of the living body, wherein the medical puncture needle comprises: a cylindrically-shaped main body possessing a central axis and a cylindrical shape, the main body including a through hole extending throughout the main body from a proximal end of the main body to a distal end of the main body; an annular-shaped blade surface that is tapered so a thickness of the main body decreases toward the distal end of the main body; and a circular-shaped blade edge at a distal-most end of the blade surface, the circular-shaped blade edge lying in a plane orthogonal to the central axis of the main body. The method additionally includes puncturing the tissue of the living body with the circular-shaped blade edge and then inserting the medical puncture needle into the tissue; and delivering the liquid medicine to the tissue, after the puncturing of the tissue, by way of the through hole in the main body.

### BRIEF DESCRIPTION OF THE DRAWINGS

FIG. 1A is a perspective view of a medical puncture needle according to a first embodiment, and FIG. 1B is a cross-sectional view of the medical puncture needle illustrated in FIG. 1A.

FIG. 2 is an explanatory diagram illustrating a function of the medical puncture needle according to the first embodiment.

FIG. 3A is an explanatory diagram (part 1) illustrating the action of a medical puncture needle of a comparative example, and FIG. 3B is an explanatory diagram (part 2) illustrating the action of the medical puncture needle of the comparative example.

FIG. 4A is a cross-sectional view (part 1) illustrating a manufacturing process of the medical puncture needle illustrated in FIG. 1A, and FIG. 4B is a cross-sectional view (part 2) illustrating the manufacturing process of the medical puncture needle.

FIG. 5A is a cross-sectional view (part 3) illustrating the manufacturing process of the medical puncture needle illustrated in FIG. 1A, and FIG. 5B is a cross-sectional view (part 4) illustrating the manufacturing process of the medical puncture needle illustrated in FIG. 1A.

FIG. 6A is a perspective view of a medical puncture needle according to a second embodiment, and FIG. 6B is a cross-sectional view of the medical puncture needle illustrated in FIG. 6A.

FIG. 7A is a perspective view of a medical puncture needle according to a third embodiment, and FIG. 7B is a cross-sectional view of the medical puncture needle illustrated in FIG. 7A.

FIG. 8A is a perspective view of a medical puncture needle according to a fourth embodiment, and FIG. 8B is a cross-sectional view of the medical puncture needle illustrated in FIG. 8A.

FIG. 9A is a perspective view of a medical puncture needle according to a fifth embodiment, and FIG. 9B is a cross-sectional view of the medical puncture needle illustrated in FIG. 9A.

### DETAILED DESCRIPTION

Set forth below with reference to the accompanying drawings is a detailed description of embodiments of a medical puncture needle representing examples of the inventive medical puncture needle disclosed here.

### First Embodiment

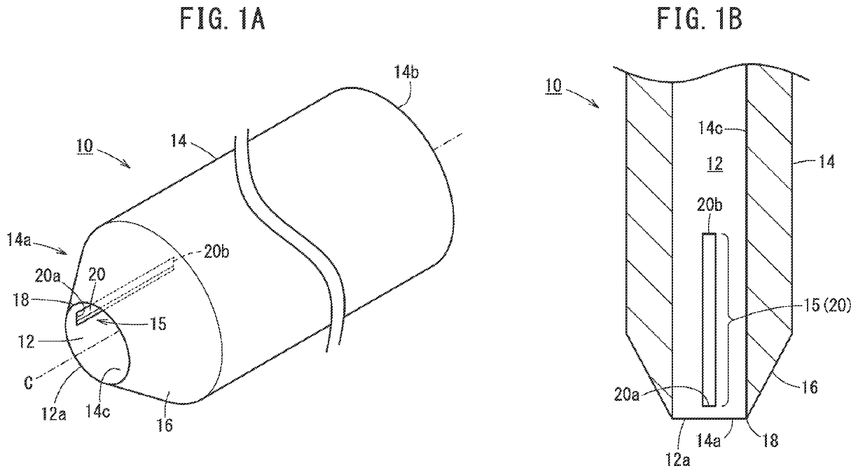

As illustrated in FIG. 1A, a medical puncture needle 10 according to the present embodiment includes a cylindrical main body 14 having a through hole 12 formed therein, a blade surface 16 provided at a needle tip 14a of the main body 14 (at a distal end portion of the main body 14), and a circular blade edge 18 formed at a tip of the blade surface 16.

The main body 14 is formed in a long (elongated) cylindrical shape and is made of, for example, a metal material such as stainless steel. The diameter (outer diameter) of the main body 14 can be, for example, about 36 G (about 0.1 mm).

The through hole 12 extends in the axial direction along a central axis C of the main body 14. The through hole 12 is formed as a cavity having a circular cross section, and penetrates axially from a proximal end 14b of the main body 14 to the needle tip 14a. When the diameter of the main body 14 is, for example, 36 G (about 0.1 mm), the inner diameter of the through hole 12 can be, for example, about 0.05 mm. An opening part 12a at the tip of the through hole 12 is provided inside the circular blade edge 18. That is, the opening part 12a of the through hole 12 is formed along a plane perpendicular to the central axis C of the medical puncture needle 10.

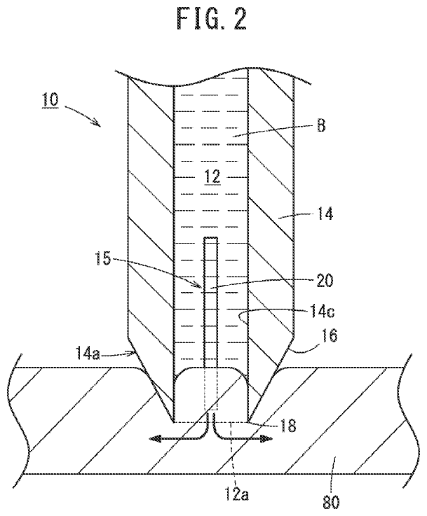

A liquid medicine permeation portion 15 is formed on an inner peripheral surface 14c of the through hole 12 near the needle tip 14a. The liquid medicine permeation portion 15 provides, and is an example of, an uneven structure on the inner peripheral surface 14c of the through hole 12. The liquid medicine permeation portion 15 of the present embodiment is in the form of a liquid medicine passage groove 20 extending in the direction of the central axis C (axial direction). The liquid medicine passage groove 20 serves as a flow path for guiding the liquid medicine to the needle tip 14a when the through hole 12 is closed by coring on a tissue 80 (see FIG. 2) due to insertion of the medical puncture needle 10 into the tissue 80. The liquid medicine passage groove 20 is formed as a groove-shaped recess that is recessed outward (outwardly away from the central axis C) with respect to the inner peripheral surface 14c, and linearly extends in the direction of the central axis C. A tip part 20a of the liquid medicine passage groove 20 is positioned in the vicinity of the tip of the through hole 12.

In addition, the liquid medicine passage groove 20 has a width and a depth capable of inhibiting entry of the tissue 80 into the liquid medicine passage groove 20 during coring. When the diameter (outer diameter) of the main body 14 is set to, for example, 36 G (about 0.10 mm) to 38 G (about 0.05 mm), the width (dimension in the circumferential direction) of the liquid medicine passage groove 20 can be set to, for example, about 0.025 to 0.070 mm, and the depth (dimension in the radial direction) of the liquid medicine passage groove 20 can be set to about 0.005 to 0.015 mm.

In addition, when the diameter (outer diameter) of the main body 14 is, for example, 22 G (about 0.70 mm) to 34 G (about 0.18 mm), the width (dimension in the circumferential direction) of the liquid medicine passage groove 20 can be, for example, about 0.060 to 0.535 mm, and the depth (dimension in the radial direction) of the liquid medicine passage groove 20 can be about 0.005 to 0.145 mm. When the liquid medicine is administered to a site where coring is likely to occur or when the liquid medicine has high viscosity, it is preferable that the diameter (outer diameter) of the main body 14 is large, the width of the liquid medicine passage groove 20 is large, and the depth of the liquid medicine passage groove 20 is small.

Further, the position of a proximal end 20b of the liquid medicine passage groove 20 illustrated in FIG. 1B preferably extends to a position where the tissue 80 (see FIG. 2) of the living body cannot enter. The proximal end 20b of the liquid medicine passage groove 20 is preferably located, for example, at a position 2.5 mm or more away from the opening part 12a toward the proximal end side.

A plurality of the liquid medicine passage grooves 20 may be provided in the inner peripheral surface 14c of the through hole 12 in the circumferential direction. In a case where two liquid medicine passage grooves 20 are provided, the liquid medicine passage grooves 20 are arranged at an angle of 180° in the circumferential direction (i.e., the two liquid medicine passage grooves 20 are diametrically opposite one another). In addition, three, four, or more liquid medicine passage grooves 20 may be provided. In a case where the plurality of liquid medicine passage grooves 20 is provided, it is preferable to arrange the liquid medicine passage grooves evenly in the circumferential direction, and the plurality of liquid medicine passage grooves can be equally spaced in the circumferential direction.

As illustrated in FIG. 1A, the blade surface 16 is formed as an inclined curved surface obtained by obliquely cutting the needle tip 14a of the cylindrical main body 14 from the outer peripheral side toward the center. As illustrated in FIG. 1B, the cross section of the main body 14 at the portion where the blade surface 16 is formed is tapered so that the thickness is decreased toward the tip. The angle (inclination angle) between the blade surface 16 and the central axis C (see FIG. 1A) of the main body 14 can be, for example, about 8° to 75°.

The blade edge 18 is sharply formed at a portion where the blade surface 16 and the inner peripheral surface 14c of the through hole 12 are in contact with each other, and can puncture the tissue 80 of the living body. The blade edge 18 is formed in an annular shape along a plane perpendicular to the central axis C of the main body 14. With this configuration, a load is distributed to the entire region of the blade edge 18 during puncturing. Therefore, the blade edge 18 is difficult to penetrate the tissue 80, so that the blade edge 18 easily punctures the tissue 80 shallowly.

The medical puncture needle 10 described above is used in such a manner that the main body 14 is inserted into the thin layered tissue 80 in a direction perpendicular to the tissue 80 (thickness direction of the tissue 80) as illustrated in FIG. 2. Examples of the thin layered tissue 80 include intestinal mucosa, posterior eye segment, and olfactory mucosa, and the thickness of the tissue 80 is 1 mm or less. When the medical puncture needle 10 is inserted in the manner described above, the entire region of the blade edge 18 is aligned in the layer direction of the thin tissue 80, so that the entire region of the opening part 12a of the through hole 12 stays in the tissue 80. With the tissue 80 being punctured by the blade edge 18, a part of the tissue 80 enters the inside of the through hole 12 by the coring. Therefore, most of the through hole 12 is closed by the tissue 80. It is to be noted, however, that the tissue 80 cannot enter the liquid medicine passage groove 20, because the liquid medicine passage groove 20 has a fine groove shape. Therefore, the liquid medicine passage groove 20 forms a flow path through which the liquid medicine can pass.

The liquid medicine is guided to the vicinity of the needle tip 14a of the medical puncture needle 10 through the liquid medicine passage groove 20, and is injected into the tissue 80 from the vicinity of the blade edge 18. As a result, the medical puncture needle 10 can efficiently administer a large amount of liquid medicine to the extremely thin tissue 80.

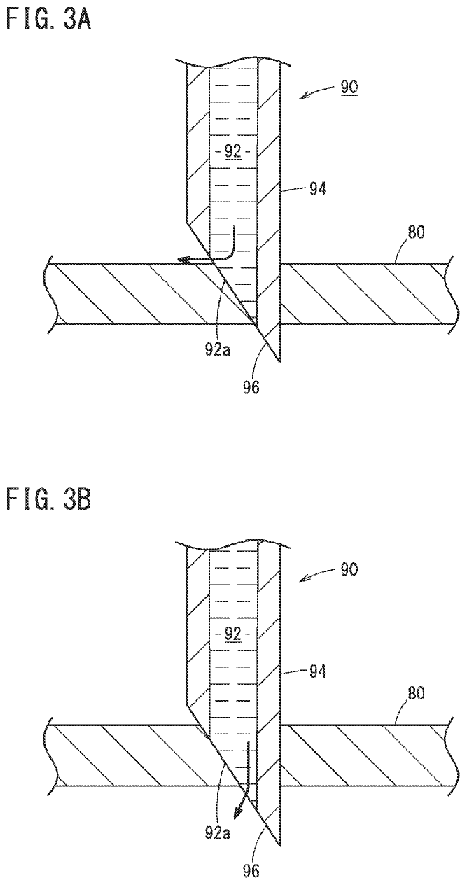

As illustrated in FIG. 3A, in a medical puncture needle 90 of a comparative example, a blade surface 96 has a plane inclined with respect to the axial direction, and an opening part 92a of a through hole 92 of a needle tube 94 is exposed to the plane inclined in the axial direction. The opening part 92a of the through hole 92 is elongated along the axial direction of the medical puncture needle 90. Therefore, when the medical puncture needle 90 is inserted, a part of the opening part 92a of the through hole 92 on the proximal end side protrudes from the thin tissue 80, and this causes a leakage of the liquid medicine.

In addition, as illustrated in FIG. 3B, when the medical puncture needle 90 of the comparative example is deeply inserted, the opening part 92a of the through hole 92 penetrates the tissue 80 to cause a leakage of the liquid medicine. Therefore, the medical puncture needle 90 of the comparative example cannot efficiently administer the liquid medicine to the thin tissue 80.

On the other hand, in the medical puncture needle 10 according to the present embodiment, the opening part 12a of the through hole 12 is formed inside the blade edge 18 perpendicular to the axial direction of the medical puncture needle 10, and thus is short in the axial direction. Therefore, even when the very thin tissue 80 is punctured, the opening part 12a does not penetrate the tissue 80, and the liquid medicine can be injected into the tissue 80 without leakage.

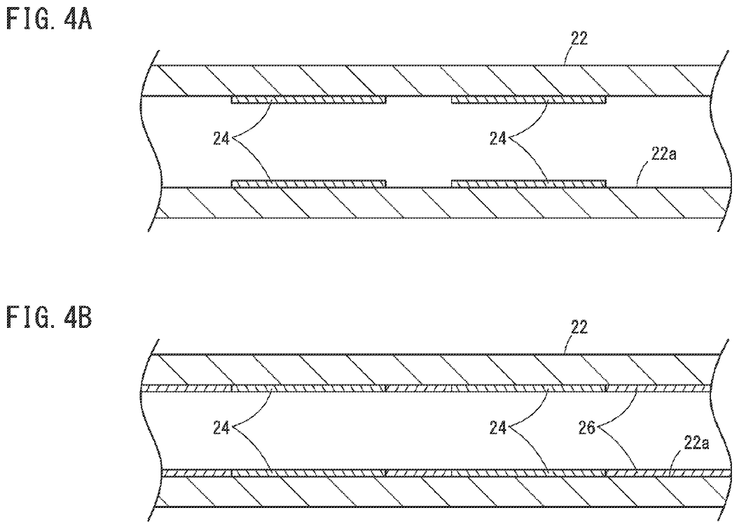

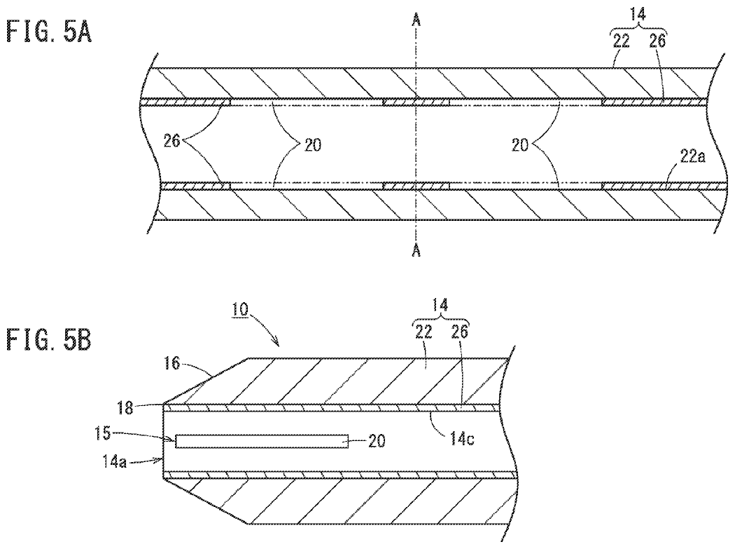

The medical puncture needle 10 described above can be manufactured in such a manner that, as illustrated in FIGS. 4A to 5B, a metal tube 22 constituting the main body 14 is prepared, and the outer peripheral portion of the needle tip 14a of the main body 14 is electrolytically polished in a tapered shape to form the blade surface 16 and the blade edge 18. In addition, the liquid medicine passage groove 20 can be formed by performing various types of processing, for example, machining (cutting or pressing), on the inner peripheral wall of the main body 14.

The liquid medicine passage groove 20 of the medical puncture needle 10 may be formed using plating. The case where the liquid medicine passage groove 20 of the medical puncture needle 10 is formed by plating will be described below.

As illustrated in FIG. 4A, first, the metal tube 22 is formed as a material of the medical puncture needle 10. Next, a mask 24 made of, for example, a resin material is formed on a part of an inner surface 22a of the metal tube 22 by coating. The mask 24 is formed in the same planar shape as the liquid medicine passage groove 20. Prior to the formation of the mask 24, a seed layer serving as a base of the plating film may be formed.

Next, as illustrated in FIG. 4B, a metal layer 26 is formed inside the metal tube 22 by a method such as electrolytic plating or electroless plating. The metal layer 26 grows with the inner surface 22a of a portion not covered with the mask 24 as a base. The inner surface 22a of the portion not covered with the mask 24 is covered with the metal layer 26. The metal layer 26 is made of, for example, metal such as Ni/Cr alloy, Cu/Cr alloy, Ni, Ni base alloy, Fe, Fe base alloy, Co, Co base alloy, Ag, Ag base alloy, Cu, Cu base alloy, Au, and Au base alloy.

Thereafter, the mask 24 is removed as illustrated in FIG. 5A. The mask 24 can be removed using a solvent, or the like. Due to the removal of the mask 24, the liquid medicine passage groove 20 having a recessed groove is formed in the metal tube 22. Next, the A-A portion in FIG. 5A is cut. Thus, two main bodies 14 having the cut portions of the metal tube 22 as tips are obtained.

Next, as illustrated in FIG. 5B, the blade surface 16 and the blade edge 18 are formed by performing electrolytic polishing obliquely along the outer periphery of the tip of the main body 14. Through the above steps, the medical puncture needle 10 according to the present embodiment is completed.

The medical puncture needle 10 according to the present embodiment has the following effects.

The medical puncture needle 10 according to the present embodiment includes: the cylindrical main body 14 having the through hole 12 formed therein; the annular blade surface 16 cut into a tapered shape so that the thickness of the main body 14 reduces toward the needle tip 14a of the main body 14; and the circular blade edge 18 formed at the tip of the blade surface 16 so as to extend along a plane orthogonal to the central axis C of the main body 14.

According to the above configuration, when the medical puncture needle 10 is inserted into the thin tissue 80 in the direction perpendicular to the tissue 80, the through hole 12 does not penetrate the tissue 80, so that a large amount of liquid medicine can be efficiently injected into the tissue 80 without causing leakage of the liquid medicine.

In the medical puncture needle 10 described above, the liquid medicine permeation portion 15 having an uneven structure may be further formed in the inner peripheral surface 14c of the through hole 12 in the vicinity of the blade edge 18.

When the medical puncture needle 10 is perpendicularly inserted into the tissue 80, the through hole 12 may be closed by the tissue 80 due to coring. Even in such a case, it is possible for the liquid medicine to flow toward the blade edge 18 through the liquid medicine permeation portion 15, whereby a sufficient amount of liquid medicine can be injected into the tissue 80.

In the medical puncture needle 10 described above, the liquid medicine permeation portion 15 may include the liquid medicine passage groove 20 having a groove-shaped recess extending in the axial direction. Accordingly, even when the through hole 12 is closed by coring, it is possible for the liquid medicine to flow toward the blade edge 18 through the liquid medicine passage groove 20.

In the medical puncture needle 10 described above, a plurality of the liquid medicine passage grooves 20 may be provided in the inner peripheral surface 14c of the through hole 12. With this configuration, even when coring occurs, a sufficient amount of the liquid medicine can be injected into the tissue 80.

### Second Embodiment

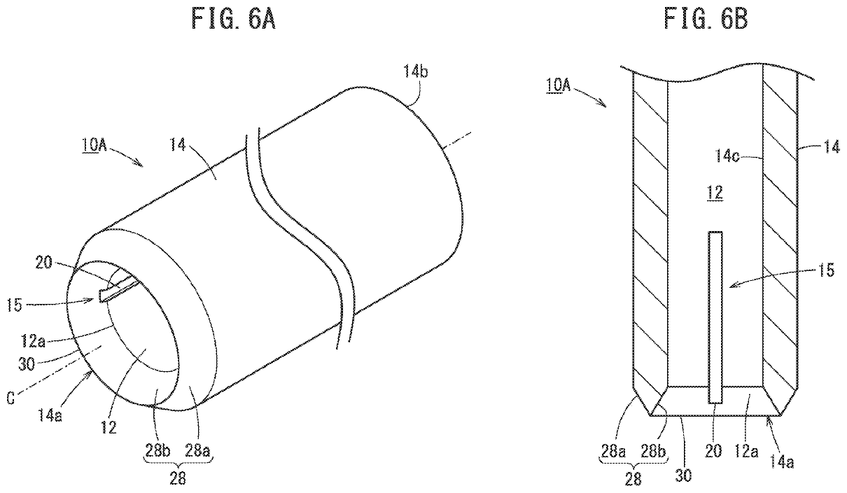

As illustrated in FIG. 6A, a medical puncture needle 10A according to the present embodiment is different from the medical puncture needle 10 in FIG. 1A in the shape of a blade surface 28 formed at a needle tip 14a of a cylindrical main body 14. The components of the medical puncture needle 10A similar to the components of the medical puncture needle 10 in FIG. 1A are denoted by the same reference numerals, and a detailed description of such components is not repeated.

As illustrated in FIG. 6B, in the medical puncture needle 10A, the blade surface 28 includes an outer blade surface 28a obtained by obliquely cutting the main body 14 from the outer peripheral side, and an inner blade surface 28b obtained by obliquely cutting the main body 14 from the inner peripheral side. The outer blade surface 28a and the inner blade surface 28b have annular curved surfaces, and a sharp blade edge 30 is formed at a tip portion where the outer blade surface 28a and the inner blade surface 28b meet. The outer blade surface 28a and the inner blade surface 28b can be formed by electrolytic polishing.

As illustrated in FIG. 6A, the blade edge 30 is formed in an annular shape (circular-shape) along a plane perpendicular to the central axis C of the main body 14. The blade edge 30 in the present embodiment is wider than the through hole 12 of the main body 14 (the diameter of the blade edge 30 is greater than the inner diameter of the through hole 12 of the main body 14), and has a larger diameter than the blade edge 18 in FIG. 1A. According to the medical puncture needle 10A of the present embodiment, a load applied during puncture of the tissue 80 is distributed to the blade edge 30 in a wider area, whereby the liquid medicine easily flows out from the gap between the blade edge 30 and the tissue 80.

As described above, in the medical puncture needle 10A according to the present embodiment, the blade surface 28 is formed on both the inner peripheral side and the outer peripheral side of the main body 14. As a result, even in a case where the coring occurs, the load applied during puncture of the tissue 80 is distributed to the blade edge 30 in a wider area, so that the liquid medicine easily flows out from the gap between the blade edge 30 and the tissue 80.

### Third Embodiment

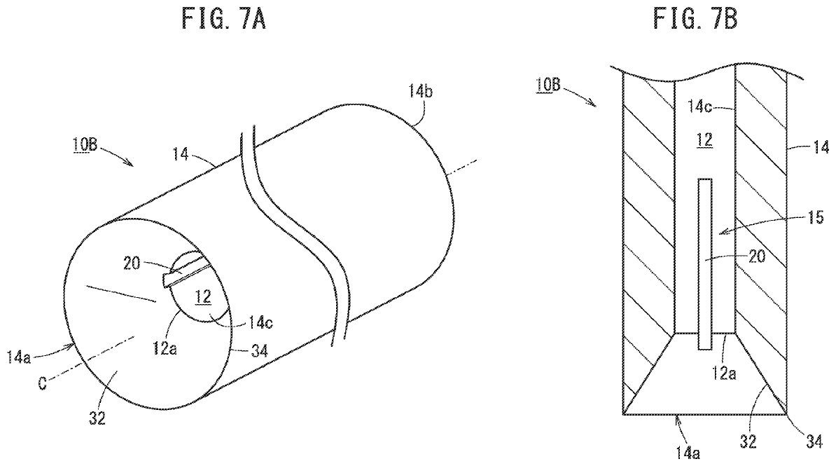

As illustrated in FIG. 7A, a medical puncture needle 10B according to the present embodiment is different from the medical puncture needle 10 in FIG. 1A in the shape of a blade surface 32 formed at a needle tip 14a of a cylindrical main body 14. Note that the components of the medical puncture needle 10B similar to the components of the medical puncture needle 10 in FIG. 1A are denoted by the same reference numerals, and the detailed description thereof will be omitted.

As illustrated in FIG. 7B, in the medical puncture needle 10B, the blade surface 32 is formed by cutting the main body 14 in a tapered shape from the inner peripheral side. The blade surface 32 has an annular curved surface obliquely cut along the circumferential direction of the main body 14. A sharp blade edge 34 is formed on a side where the outer peripheral surface of the main body 14 and the blade surface 32 meet. The blade edge 34 in the present embodiment is wider than the through hole 12 of the main body 14 (the diameter of the blade edge 34 is greater than the inner diameter of the through hole 12 of the main body 14), and has a diameter equal to the diameter of the main body 14.

As described above, in the medical puncture needle 10B according to the present embodiment, the blade surface 32 is formed only on the inner peripheral side of the main body 14. Thus, the blade edge 34 widens to have a diameter substantially equal to the diameter of the main body 14. As a result, even in a case where the coring occurs when the medical puncture needle 10B is inserted into the tissue 80, the load applied during insertion into the tissue 80 is distributed to the blade edge 34 in a wider area, so that the liquid medicine easily flows out from the gap between the blade edge 34 and the tissue 80. As a result, the medical puncture needle 10B can inject a large amount of liquid medicine into the tissue 80.

### Fourth Embodiment

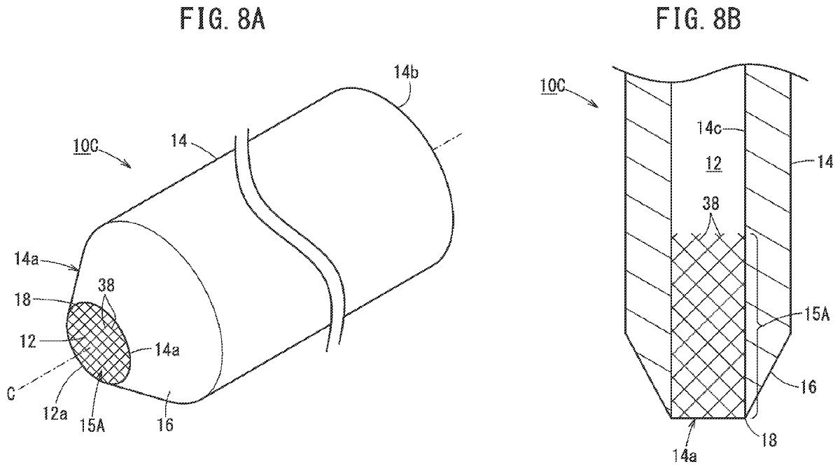

As illustrated in FIGS. 8A and 8B, a medical puncture needle 10C according to the present embodiment is different from the medical puncture needle 10 in FIG. 1A in a liquid medicine permeation portion 15A formed in an inner peripheral surface 14c of a through hole 12 of a main body 14. Components of the medical puncture needle 10C similar to components of the medical puncture needle 10 in FIG. 1A are denoted by the same reference numerals, and a detailed description of such components is not repeated.

In the medical puncture needle 10C according to the present embodiment, the liquid medicine permeation portion 15A having an uneven structure is formed in the inner peripheral surface 14c of the through hole 12. The liquid medicine permeation portion 15A is formed by a plurality of liquid medicine passage grooves 38 arranged in a matrix. In the illustrated embodiment, the liquid medicine passage grooves 38 arranged in the matrix cross/intersect one another. The liquid medicine passage grooves 38 can be formed by changing the pattern of the mask 24 in the step of forming the liquid medicine passage groove 20 by plating described with reference to FIGS. 4A to 5B.

According to the medical puncture needle 10C of the present embodiment, even if coring occurs during puncture of the tissue 80, it is possible for the liquid medicine to flow toward the needle tip 14a of the main body 14 through the liquid medicine permeation portion 15A. As a result, the medical puncture needle 10C can inject a large amount of liquid medicine into the tissue 80.

The structure of the liquid medicine permeation portion 15A of the present embodiment is not limited to the liquid medicine passage grooves 38 in a matrix, and can be replaced with various uneven structures such as protrusions that can generate a gap between the tissue 80 and the inner peripheral surface 14c of the through hole 12 when coring occurs.

### Fifth Embodiment

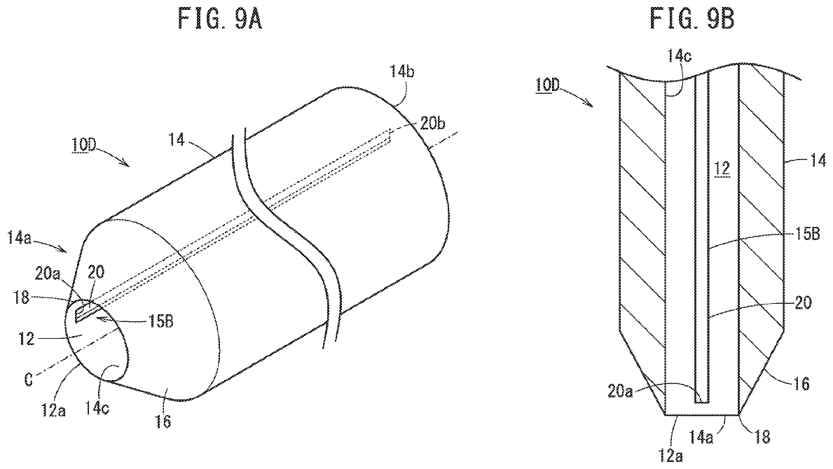

As illustrated in FIGS. 9A and 9B, a medical puncture needle 10D according to the present embodiment is different from the medical puncture needle 10 in FIG. 1A in a liquid medicine permeation portion 15B formed in an inner peripheral surface 14c of a through hole 12 of a main body 14. Components of the medical puncture needle 10D similar to components of the medical puncture needle 10 in FIG. 1A are denoted by the same reference numerals, and a detailed description of such components is not repeated.

The medical puncture needle 10D according to the present embodiment has the liquid medicine permeation portion 15B which is formed in the inner peripheral surface 14c of the through hole 12 and which has a liquid medicine passage groove 20 extending to a proximal end 14b of the medical puncture needle 10D. The medical puncture needle 10D according to the present embodiment is manufactured as follows. First, a core material having the same outer peripheral surface shape as the shape of the inner peripheral surface 14c of the through hole 12 and the liquid medicine permeation portion 15B is prepared.

The core material is formed with a linear protrusion extending in the axial direction in a portion corresponding to the liquid medicine passage groove 20. Next, the core material is immersed in a plating solution (containing, for example, ions of Ni/Cr, Cu/Cr, Ni, Fe, Co, Ag, Cu, Au, etc.), and an electroformed body (for example, an electroformed body of Ni/Cr, Cu/Cr, Ni, Fe, Co, Ag, Cu, Au, etc.) is formed on the outer peripheral surface of the core material by plating. An electrolytic plating method is preferably used for forming the electroformed body by plating, but an electroless plating method may be partly used in combination during the process. Thereafter, the core material is removed from the electroformed body from the proximal end side to obtain the main body 14 constituted by a tubular electroformed body. The liquid medicine passage groove 20 is formed in the inner peripheral surface 14c of the main body 14. Then, the blade surface 16 and the blade edge 18 are formed by performing electrolytic polishing obliquely along the outer periphery of the tip of the main body 14. Through the above steps, the medical puncture needle 10D according to the present embodiment is completed.

The medical puncture needles in the second embodiment and the third embodiment described above can also be manufactured in the same manner.

The detailed description above describes embodiments of a medical puncture needle for administering a medicine into a living body representing examples of the inventive medical puncture needle disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical puncture needle comprising:
a main body possessing a central axis and a cylindrical shape, the main body including a through hole extending throughout the main body from a proximal end of the main body to a distal end of the main body;
a blade surface that has an annular shape and that is tapered so that a thickness of the main body decreases toward the distal end of the main body; and
a blade edge that has an annular shape, the blade edge being at a tip of the blade surface and being configured so that the blade edge extends along a plane orthogonal to the central axis of the main body.

2. The medical puncture needle according to claim 1, wherein the main body includes an inner peripheral surface surrounding the through hole, and further comprising a liquid medicine permeation portion formed on the inner peripheral surface of the through hole and terminating adjacent the blade edge, the liquid medicine permeation portion having an uneven structure.

3. The medical puncture needle according to claim 2, wherein the liquid medicine permeation portion includes a liquid medicine passage configured as a groove-shaped recess extending in an axial direction.

4. The medical puncture needle according to claim 3, wherein the inner peripheral surface of the main body includes a plurality of the liquid medicine passages.

5. The medical puncture needle according to claim 1, wherein the blade surface is provided on an outer peripheral side of the main body.

6. The medical puncture needle according to claim 1, wherein the blade surface is provided on both an inner peripheral side and an outer peripheral side of the main body.

7. The medical puncture needle according to claim 1, wherein the blade surface is provided only on an inner peripheral side of the main body.

8. A medical puncture needle for puncturing tissue of a living body to administer liquid medicine, the medical puncture needle comprising:
a cylindrically-shaped main body possessing a central axis, the main body including a through hole that extends axially throughout the main body from a proximal end of the cylindrically-shaped main body to an opposite axial end of the cylindrically-shaped main body, the main body including an inner surface that surrounds the through hole;
the main body including a distal end portion that terminates at the distal end of the main body, an annular-shaped inclined surface at the distal end portion of the main body, the inclined surface being inclined relative to the central axis so that the distal end portion of the main body is tapered and a thickness of the main body decreases toward the distal end of the main body;
the inclined surface terminating at a blade edge that is first brought into contact with the tissue of the living body during the puncture of the tissue of the living body to administer the liquid medicine; and
a liquid medicine groove in the inner surface of the main body defining a flow path for guiding the liquid medicine to the distal end portion of the main body when the through hole is closed as a result of coring during the puncture of the tissue.

9. The medical puncture needle according to claim 8, wherein the blade edge is a circular-shaped blade edge, the circular-shaped blade edge lying in a plane that is perpendicular to the central axis.

10. The medical puncture needle according to claim 8, wherein the liquid medicine groove is an axially extending liquid medicine groove, the axially extending liquid medicine groove possessing a distal-most end that is spaced proximally from the circular-shaped blade edge.

11. The medical puncture needle according to claim 8, wherein the liquid medicine groove is an axially extending liquid medicine groove, the axially extending liquid medicine groove possessing a proximal-most end that is spaced distally from the proximal end of the main body.

12. The medical puncture needle according to claim 8, wherein the inner peripheral surface of the main body includes a plurality of the liquid medicine passages.

13. The medical puncture needle according to claim 8, wherein the inclined surface is an outer peripheral surface of the main body.

14. The medical puncture needle according to claim 8, wherein the inclined surface is an inner peripheral surface of the main body.

15. The medical puncture needle according to claim 8, wherein the inclined surface is an outer peripheral surface of the main body, a distal portion of the inner peripheral surface of the main body also being an inclined surface that is inclined relative to the central axis.

16. The medical puncture needle according to claim 8, wherein the liquid medicine groove is one of plural liquid medicine grooves that intersect one another to form a matrix.

17. A method of administering liquid medicine to tissue of a living body, the method comprising:
moving a medical puncture needle toward the tissue of the living body, the medical puncture needle comprising:
a cylindrically-shaped main body possessing a central axis and a cylindrical shape, the main body including a through hole extending throughout the main body from a proximal end of the main body to a distal end of the main body;
an annular-shaped blade surface that is tapered so a thickness of the main body decreases toward the distal end of the main body; and
a circular-shaped blade edge at a distal-most end of the blade surface, the circular-shaped blade edge lying in a plane orthogonal to the central axis of the main body;
puncturing the tissue of the living body with the circular-shaped blade edge and then inserting the medical puncture needle into the tissue; and
delivering the liquid medicine to the tissue, after the puncturing of the tissue, by way of the through hole in the main body.

18. The method according to claim 17, the main body including an inner surface that surrounds the through hole, and a liquid medicine groove in the inner surface of the main body defining a flow path, the method further comprising guiding the liquid medicine in the through hole toward the distal end of the main body by flowing the liquid medicine through the liquid medicine groove.

19. The method according to claim 18, wherein the liquid medicine groove possesses a distal-most end that is spaced proximally from the circular-shaped blade edge.

20. The method according to claim 18, wherein the liquid medicine groove possesses a proximal-most end that is spaced distally from the proximal end of the main body.
